(19) United States
(12) Patent Application Publication
(10) Pub. No.: US 2025/0302425 A1
(43) Pub. Date: Oct. 2, 2025

(54) ELECTRONIC STETHOSCOPE

(71) Applicant: Murata Manufacturing Co., Ltd., Kyoto (JP)

(72) Inventors: Hiroki Tsuchiya, Kyoto (JP); Hirofumi Tsuchimoto, Kyoto (JP); Koji Tanaka, Kyoto (JP)

(21) Appl. No.: 19/092,348

(22) Filed: Mar. 27, 2025

(30) Foreign Application Priority Data
Mar. 29, 2024 (JP) 2024-056766

(51) Int. Cl. A61B 7/04; A61B 7/00; H04R 1/46

(52) U.S. Cl. CPC A61B 7/04; A61B 7/003; H04R 1/46

(57) ABSTRACT

An electronic stethoscope includes a diaphragm that comes into contact with a living body, a sound sensor that receives vibration from the diaphragm and converts the vibration into an electric signal, and a tubular member that includes an inner wall surface to define an internal space. The tubular member includes a first inner wall surface that defines a first space, a second inner wall surface that defines a second space, and a boundary portion where the first inner wall surface is in contact with the second inner wall surface. A volume of the first space is larger than a volume of the second space. A cross-sectional area of a transverse section of the internal space is gradually decreased from a diaphragm side end toward the boundary portion in the first space and is gradually increased from the boundary portion toward a sound sensor side end in the second space.

### CROSS REFERENCE TO RELATED APPLICATION

This application claims priority from Japanese Patent Application No. 2024-056766 filed on Mar. 29, 2024. The content of this application is incorporated herein by reference in its entirety.

### BACKGROUND

The present disclosure relates to an electronic stethoscope.

For example, Japanese Unexamined Patent Application Publication No. 2022-119446 describes an electronic stethoscope which includes a diaphragm that comes into contact with a living body, a microphone (a sound sensor) that receives vibration propagated from the diaphragm and converts the vibration into an electric signal, and a chestpiece that is provided with an internal space to propagate the vibration from the diaphragm toward the microphone.

### BRIEF SUMMARY

However, the electronic stethoscope described in Japanese Unexamined Patent Application Publication No. 2022-119446 cannot sufficiently pick up a living body sound having a low sound pressure level such as a pulmonary sound.

The present disclosure sufficiently picks up a living body sound having a low sound pressure level with an electronic stethoscope configured to collect a living body sound.

An aspect of the present disclosure to solve the above technical problem provides an electronic stethoscope including:
a diaphragm including
   a first surface that comes into contact with a living body, and
   a second surface located on an opposite side of the first surface;
a sound sensor that receives vibration propagated from the diaphragm and converts the vibration into an electric signal; and
a tubular member that includes an inner wall surface to define an internal space in which the vibration is propagated from the diaphragm toward the sound sensor, in which
the inner wall surface of the tubular member includes
   a first inner wall surface that defines a first space on a diaphragm side,
   a second inner wall surface that defines a second space on a sound sensor side, and
   a boundary portion where the first inner wall surface is in contact with the second inner wall surface,
a volume of the first space is larger than a volume of the second space, and
a cross-sectional area of a transverse section of the internal space of the tubular member intersecting with a direction of extension of the internal space is gradually decreased from an end on the diaphragm side toward the boundary portion in the first space and is gradually increased from the boundary portion toward an end on the sound sensor side in the second space.

According to the present disclosure, an electronic stethoscope configured to collect a living body sound can sufficiently pick up a living body sound having a low sound pressure level.

### BRIEF DESCRIPTION OF THE DRAWINGS

FIG. 1 is a schematic perspective view of an electronic stethoscope according to Embodiment 1 of the present disclosure;

FIG. 2 is a cross-sectional view of the electronic stethoscope taken along the A-A line in FIG. 1;

FIG. 3 is an exploded perspective view of the electronic stethoscope according to the Embodiment 1;

FIG. 4 is a cross-sectional view of an electronic stethoscope according to Embodiment 2;

FIG. 5 is an enlarged cross-sectional view around a second space in the electronic stethoscope according to the Embodiment 2;

FIG. 6 is a cross-sectional view of an electronic stethoscope according to Embodiment 3;

FIG. 7 is a cross-sectional view of an electronic stethoscope according to Embodiment 4;

FIG. 8 is a cross-sectional view of an electronic stethoscope according to another example of the Embodiment 4;

FIG. 9 is a cross-sectional view of an electronic stethoscope according to Embodiment 5; and

FIG. 10 is an exploded perspective view of the electronic stethoscope according to the Embodiment 5.

### DETAILED DESCRIPTION

Embodiments of the present disclosure will be described below with reference to the drawings.

### Embodiment 1

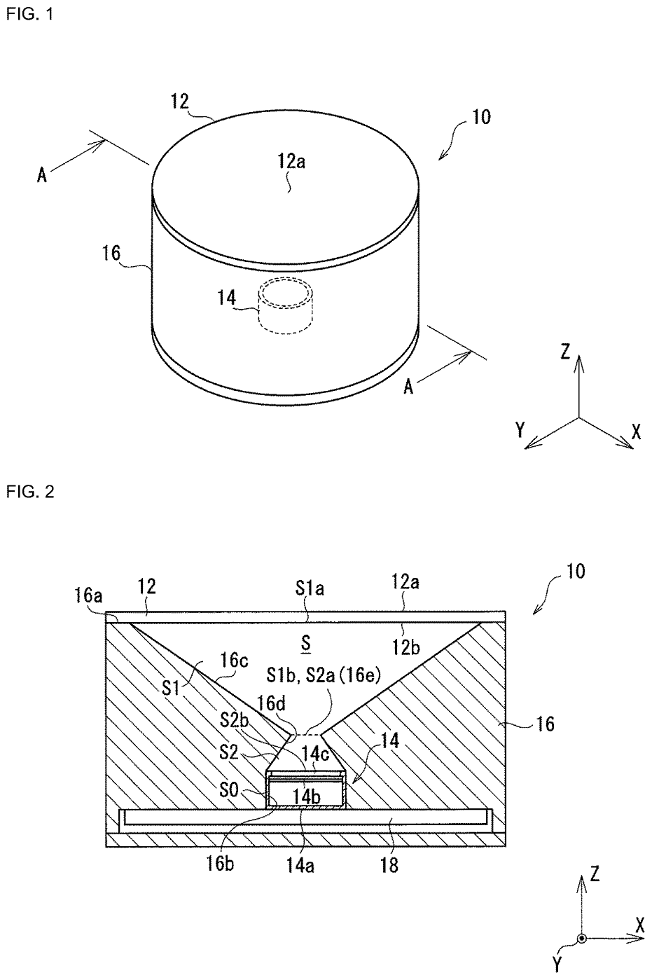

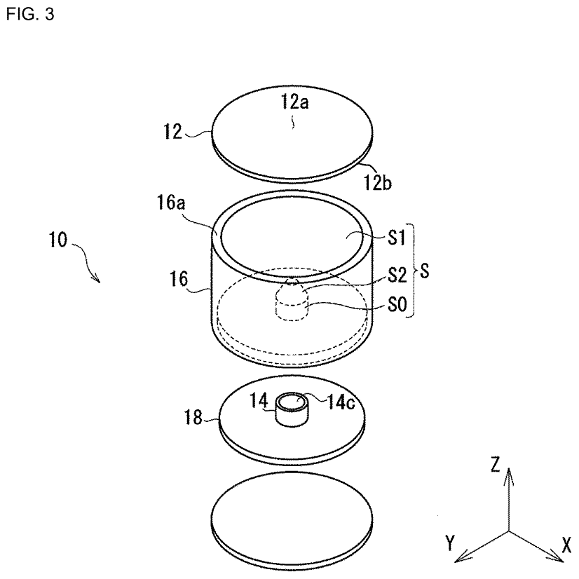

FIG. 1 is a schematic perspective view of an electronic stethoscope according to Embodiment 1 of the present disclosure. Meanwhile, FIG. 2 is a cross-sectional view of the electronic stethoscope according to the Embodiment 1, which is taken along the A-A line shown in FIG. 1. Moreover, FIG. 3 is an exploded perspective view of the electronic stethoscope according to the Embodiment 1. Note that the x-y-z orthogonal coordinate system shown in the drawings is provided in order to facilitate the understanding of the embodiments of the present disclosure and is not intended to restrict the embodiments. Here, the x axis direction indicates a width direction of the electronic stethoscope, the y axis direction indicates a depth direction thereof, and the z axis direction indicates a thickness direction thereof. In the meantime, the z axis direction is a direction in which the electronic stethoscope comes into contact with a living body.

An electronic stethoscope 10 according to the Embodiment 1 shown in FIG. 1 is an electronic device that collects a living body sound generated from a living body such as a human in a state of contact with the living body. As shown in FIGS. 1 to 3, the electronic stethoscope 10 includes a diaphragm 12 that comes into contact with the living body, and a sound sensor 14 that receives vibration propagated from the diaphragm 12 and converts the vibration into an electric signal. Moreover, the electronic stethoscope 10 includes a housing 16 that houses the sound sensor 14 while supporting the diaphragm 12.

The diaphragm 12 is a flexible sheet-like member formed from an elastic material. In the case of the present Embodiment 1, the diaphragm 12 has a circular shape when viewed in the thickness direction (the z axis direction) thereof. Meanwhile, the diaphragm 12 includes a first surface 12a that comes into contact with the living body, and a second surface 12b located on an opposite side of the first surface 12a. When the diaphragm 12 comes into contact with the living body, the diaphragm 12 is vibrated at such a frequency and an amplitude that correspond to a living body sound (such as a pulmonary sound) generated by the living body.

The sound sensor 14 is provided in the housing 16 and is configured to receive the vibration (namely, the living body sound) propagated from the diaphragm 12 into the housing 16 and to convert the vibration into the electric signal (living body sound data). The sound sensor 14 is a microphone, for example.

As shown in FIG. 2, the sound sensor 14 includes a casing 14a and a vibration plate 14b disposed in the casing 14a, for example. The casing 14a is provided with a sound collection port 14c for taking the vibration propagated from the diaphragm 12 into the casing 14a. The vibration plate 14b of the sound sensor 14 receives the vibration propagated from the diaphragm 12 via the sound collection port 14c of the casing 14a, and the vibration plate 14b is then vibrated.

Here, conversion from the living body sound to the electric signal (that is to say, conversion from the vibration of the vibration plate 14b to the electric signal) by the sound sensor 14 involves an electrodynamic mode, an electrostatic mode, a piezoelectric mode, and so forth. In the embodiments of the present disclosure, such a method of conversion from the vibration of the vibration plate 14b to the electric signal is not limited.

Meanwhile, in the case of the present Embodiment 1, the sound sensor 14 is mounted on a circuit board 18, and is housed in the housing 16 together with the circuit board 18.

For example, the electric signal (the living body sound data) outputted from the sound sensor 14 is outputted as a sound through an earphone, a speaker, and the like. Alternatively, the electric signal is transmitted to an external device by the intermediary of a radio communication device (not shown) mounted on the circuit board 18, for example. In the embodiments of the present disclosure, an output destination and usage of the living body sound data are not limited.

The housing 16 is a so-called chestpiece, which is a portion of the electronic stethoscope 10 to be held by a user such as a doctor in use. The housing 16 is formed from a rigid material and has a columnar shape in the case of the present Embodiment 1. Moreover, the housing 16 includes an end surface 16a to which the diaphragm 12 is attached. For example, the diaphragm 12 is fixed to the end surface 16a of the housing 16 by using an adhesive, a double sided tape, and the like. Alternatively, an annular cover member is attached to the housing 16 in such a way as to cover an outer peripheral edge at the first surface 12a of the diaphragm 12 placed on the end surface 16a of the housing 16, and the diaphragm 12 is fixed to the housing 16 as a consequence.

Meanwhile, in the case of the present Embodiment 1, the housing 16 includes an internal space S that extends in a height direction (the z axis direction). The internal space S is open at the end surface 16a to which the diaphragm 12 is attached. Accordingly, the second surface 12b of the diaphragm 12 being fixed to the end surface 16a faces the internal space S. As a consequence, the vibration of the diaphragm 12 is propagated to the internal space S.

In the case of the present Embodiment 1, the sound sensor 14 is housed in the internal space S of the housing 16. Thus, the internal space S communicates with an internal space of the casing 14a of the sound sensor 14, and the vibration from the diaphragm 12 is propagated to the vibration plate 14b in the sound sensor 14 via the internal space S of the housing 16. As a consequence, the sound sensor 14 can collect the living body sound from the living body in contact with the first surface 12a of the diaphragm 12 via the diaphragm 12 and the internal space S of the housing 16.

Meanwhile, the internal space S of the housing 16 is configured to amplify a sound pressure level (an amplitude of the vibration) of the living body sound to be propagated from the housing 16 to the sound sensor 14.

To be more precise, in the case of the present Embodiment 1, the internal space S of the housing 16 includes three spaces S0, S1, and S2.

The space S0 in the internal space S of the housing 16 is a sound sensor housing space for housing the sound sensor 14. In the case of the present Embodiment 1, the sound sensor housing space S0 is a cylindrical space corresponding to the cylindrical casing 14a of the sound sensor 14, which is defined by an inner wall surface 16b of the housing 16.

A first space S1 in the internal space S of the housing 16 is a space portion located closest to the diaphragm 12, which is open at the end surface 16a of the housing 16. In other words, a diaphragm side end Sla of the first space S1 faces the diaphragm 12.

Meanwhile, in the case of the present Embodiment 1, the first space S1 is a space having a truncated conical shape in which a cross-sectional area of a transverse section thereof is gradually decreased from the diaphragm side end Sla toward a second space side end S1b, and this space is defined by an inner wall surface 16c (a first inner wall surface) of the housing 16. Here, the “transverse section” cited in the present specification means a cross-section of the internal space S intersecting with a direction of extension (the z axis direction in the case of the present Embodiment 1) of the internal space S. In the case of the present Embodiment 1, the inner wall surface 16c extends linearly when viewed in the directions (the x axis direction and the y axis direction) intersecting with the direction of extension of the internal space S.

In the case of the present Embodiment 1, a first space side end S2a of the second space S2 in the internal space S of the housing 16 is connected to the second space side end S1b of the first space S1, and a sound sensor side end S2b thereof is connected to the sound sensor housing space S0. In other words, the inner wall surface of the housing 16 that defines the internal space S includes a boundary portion 16e where the inner wall surface 16c defining the first space S1 is in contact with an inner wall surface 16d defining the second space S2.

On the other hand, in the case of the present Embodiment 1, the second space S2 in the internal space S of the housing 16 is a space having a truncated conical shape in which a cross-sectional area of a transverse section thereof is gradually increased from the first space side end S2a toward the sound sensor side end S2b, and this space is defined by the inner wall surface 16d (a second inner wall surface) of the housing 16. In the case of the present Embodiment 1, the inner wall surface 16d extends linearly when viewed in the directions (the x axis direction and the y axis direction) intersecting with the direction of extension of the internal space S.

In other words, the cross-sectional area of the transverse section of the internal space S between the diaphragm 12 and the sound sensor 14 is once gradually decreased toward the sound sensor 14 and is increased thereafter.

Meanwhile, a volume of the first space S1 is larger than a volume of the second space S2. Here, the volumes can be obtained by calculation and also by filling the spaces with a fluid and measuring a filled amount thereof.

Moreover, in the case of the present Embodiment 1, the cross-sectional area of the transverse section at the diaphragm side end Sla of the first space S1 is larger than the cross-sectional area of the transverse section at the sound sensor side end S2b of the second space S2. Furthermore, in the case of the present Embodiment 1, a distance between the diaphragm side end Sla and the second space side end S1b of the first space S1 is larger than a distance between the first space side end S2a and the sound sensor side end S2b of the second space S2 in terms of the direction of extension (the z axis direction in the case of the present Embodiment 1) of the internal space S. In addition, the cross-sectional area of the transverse section at the boundary portion 16e where the inner wall surface 16c defining the first space S1 is in contact with the inner wall surface 16d defining the second space S2 is smaller than an opening area of the sound collection port 14c of the sound sensor 14.

According to the internal space S of the housing 16 having the above-described shape, or to the first and second spaces S1 and S2, in particular, a sound pressure level of a faint living body sound which is generated from a living body in contact with the first surface 12a of the diaphragm 12 is amplified and the living body sound is efficiently transmitted to (collected with) the sound sensor 14.

In a case where the second space S2 is absent unlike the case of the present Embodiment 1 and the cross-sectional area of the transverse section at the sound sensor side end of the first space S1 is smaller than the opening area of the sound collection port 14c of the sound sensor 14, the space rapidly expands at the sound collection port 14c, thus leading to reduction in acoustic energy, or reduction in sound pressure level in other words.

On the other hand, in the case where the second space S2 is absent unlike the case of the present Embodiment 1 and the cross-sectional area of the transverse section at the sound sensor side end of the first space S1 is larger than the opening area of the sound collection port 14c of the sound sensor 14, it is not possible to sufficiently increase a density of the acoustic energy before the sound reaches the sound sensor 14. In other words, the sound pressure level cannot be sufficiently amplified.

Accordingly, in the case of the present Embodiment 1, the second space S2 having the cross-sectional area of the transverse section that is gradually increased toward the sound sensor 14 is provided between the first space S1 and the sound sensor 14. Thus, the sound sensor 14 can output the electric signal corresponding to the living body sound at the amplified sound pressure level as compared to the case where the second space S2 is absent or in comparison with a case where the transverse section of the second space S2 is uniform. As a consequence, the sound pressure level of the faint living body sound is amplified and the sound is efficiently transmitted to (collected with) the sound sensor 14.

In consideration of the above-described effect, it is preferable to determine the shape of the second space S2 based on a size of the sound collection port 14c of the sound sensor 14. To be more precise, it is preferable to determine the shape of the second space S2 such that the size of the transverse section at the sound sensor side end S2b of the second space S2 substantially coincides with the size of the sound collection port 14c of the sound sensor 14.

According to the present Embodiment 1 as described above, the electronic stethoscope 10 configured to collect a living body sound can sufficiently pick up a living body sound having a low sound pressure level.

### Embodiment 2

The present Embodiment 2 is a modified embodiment of the above-described Embodiment 1, and the second space S2 in the internal space S of the housing is different from that of the Embodiment 1. Accordingly, a description will be given of the present Embodiment 2 while focusing on the different features. Note that constituents that are substantially the same as the constituents of the above-described Embodiment 1 will be denoted by the same reference signs.

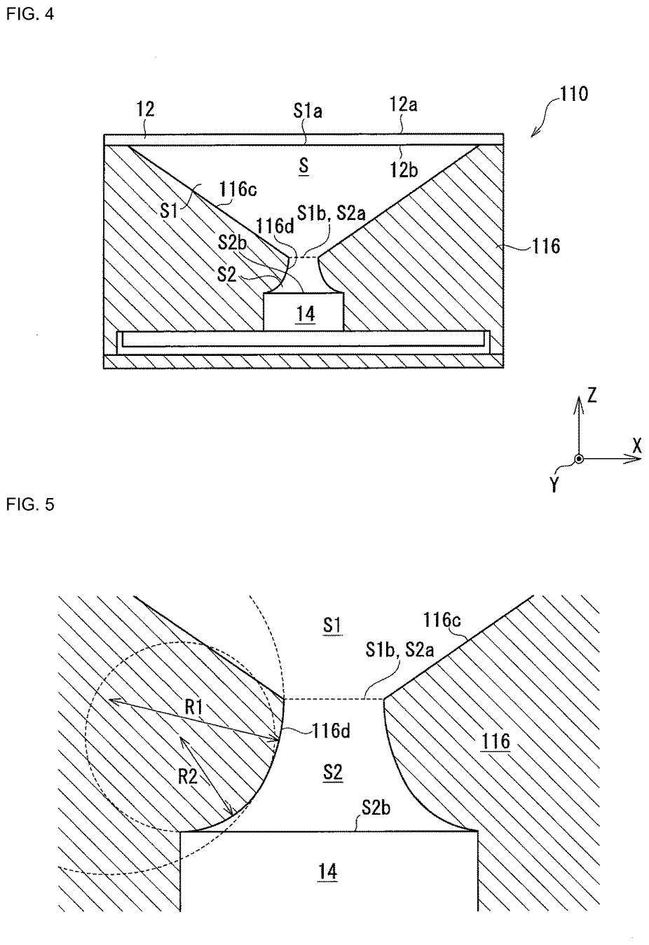

FIG. 4 is a cross-sectional view of an electronic stethoscope according to the Embodiment 2.

As shown in FIG. 4, in an electronic stethoscope 110 according to the present Embodiment 2, a second space S2 in an internal space S of a housing 116 is a space having a shape in which a cross-sectional area of a transverse section thereof is gradually increased from the first space side end S2a toward the sound sensor side end S2b, and this space is defined by an inner wall surface 116d (the second inner wall surface). Meanwhile, in the case of the present Embodiment 2, the inner wall surface 116d is convexly curved when viewed in the directions (the x axis direction and the y axis direction) intersecting with the direction of extension of the internal space S. In other words, the inner wall surface 116d has a horn shape.

According to the second space S2 in the internal space S of the housing 116 of the present Embodiment 2, a sound pressure level of a faint living body sound which is generated from a living body in contact with the first surface 12a of the diaphragm 12 is amplified and the living body sound is efficiently transmitted to (collected with) the sound sensor 14. To be more precise, due to a horn effect attributed to the horn shape of the second space S2, directivity of the acoustic energy is improved as compared to that of the truncated conical shaped second space S2 of the housing 16 of the above-described Embodiment 1, and the acoustic energy is transmitted to the sound sensor 14 more efficiently.

Here, it is preferable that a curvature radius of the curved inner wall surface 116d defining the horn-shaped second space S2 be gradually decreased from the first space side end S2a toward the sound sensor 14.

FIG. 5 is an enlarged cross-sectional view around the second space in the electronic stethoscope according to the Embodiment 2.

As shown in FIG. 5, a portion close to the first space side end S2a out of the inner wall surface 116d of the housing 116 that defines the second space S2 is a curved surface having a curvature radius R1. A portion close to the sound sensor side end S2b out of the inner wall surface 116d is a curved surface having a curvature radius R2 that is smaller than the curvature radius R1. The portion close to the first space side end S2a having the small curvature radius is a portion of the inner wall surface 116d within one-third of an extended length of the second space S2 from the first space side end S2a (a boundary portion in contact with inner wall surfaces 116c and 116d), for example. Meanwhile, the portion close to the sound sensor side end S2b is a portion of the inner wall surface 116d within one-third of the extended length of the second space S2 from the sound sensor side end S2b, for example. In this way, a larger horn effect is available from the second space S2. In other words, the directivity of the acoustic energy is further improved whereby the acoustic energy can be transmitted to the sound sensor 14 even more efficiently.

According to the present Embodiment 2 as described above, the electronic stethoscope 110 configured to collect a living body sound can sufficiently pick up a living body sound having a low sound pressure level as with the above-described Embodiment 1.

### Embodiment 3

The present Embodiment 3 is a modified embodiment of the above-described Embodiment 2, and the first space S1 in the internal space S of the housing is different from that of the Embodiment 2. Accordingly, a description will be given of the present Embodiment 3 while focusing on the different features. Note that constituents that are substantially the same as the constituents of the above-described Embodiment 2 will be denoted by the same reference signs.

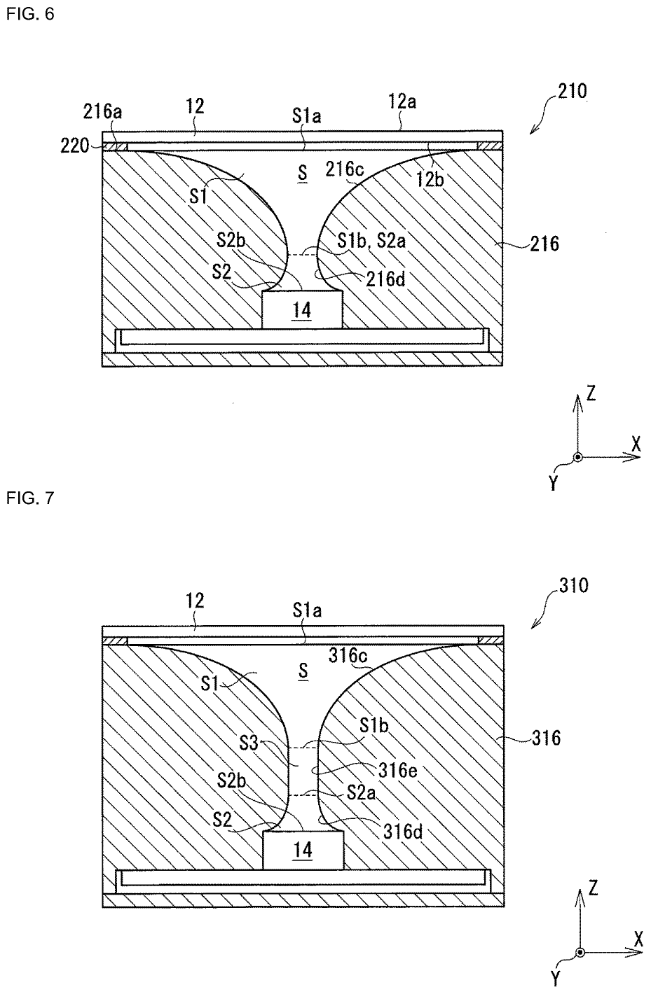

FIG. 6 is a cross-sectional view of an electronic stethoscope according to the Embodiment 3.

As shown in FIG. 6, in an electronic stethoscope 210 according to the present Embodiment 3, a first space S1 in an internal space S of a housing 216 is a space having a shape in which a cross-sectional area of a transverse section thereof is gradually decreased from the diaphragm side end Sla toward the second space side end S1b, and this space is defined by an inner wall surface 216c (the first inner wall surface). Meanwhile, in the case of the present Embodiment 3, the inner wall surface 216c is convexly curved when viewed in the directions (the x axis direction and the y axis direction) intersecting with the direction of extension of the internal space S.

According to the first space S1 in the internal space S of the housing 216 of the present Embodiment 3, a sound pressure level of a faint living body sound which is generated from a living body in contact with the first surface 12a of the diaphragm 12 is further amplified. To be more precise, as compared to the truncated conical shaped first space S1 of the housing 16 of the above-described Embodiment 1, the first space S1 of the housing 216 according to the present Embodiment 3 can further increase the density of the acoustic energy whereby the sound pressure level is further increased.

Here, depending on a curvature radius of the inner wall surface 216c of the housing 216 that defines the first space S1, an elastic modulus of the diaphragm 12, and so forth, the inner wall surface 216c may come into contact with the second surface 12b of the diaphragm 12. In other words, the diaphragm 12 continues flexural deformation as a consequence of continuous contact of the first surface 12a with the living body, whereby the second surface 12b continues contact with the inner wall surface 216c of the housing 216. The occurrence of the above-described contact leads to restriction of the vibration of the diaphragm 12.

Accordingly, in the case of the present Embodiment 3, an annular spacer member 220 is provided between the outer peripheral edge at the second surface 12b of the diaphragm 12 and an end surface 216a of the housing 216. The second surface 12b of the diaphragm 12 is located sufficiently away from the inner wall surface 216c of the housing 216 by using this spacer member 220, and the second surface 12b of the diaphragm 12 is kept from coming into contact with inner wall surface 216c of the housing 216. This makes it possible to suppress changes in characteristics of the acoustic energy directed from the diaphragm 12 to the sound sensor 14, which may be caused by restriction of vibration strokes of the diaphragm 12 due to the contact with the inner wall surface 216c.

Here, an internal space (a space where the vibration is propagated) of the annular spacer member 220 is not limited to be of a cylindrical shape but may be of a truncated conical shape instead. Meanwhile, the spacer member 220 and the housing 216 may be integrated into a single component.

In the meantime, an inner wall surface 216d that defines the second space S2 in the internal space S of the housing 216 may extend linearly when viewed in the directions (the x axis direction and the y axis direction) intersecting with the direction of extension of the internal space S as with the second space S2 in the housing 16 according to the Embodiment 1.

According to the present Embodiment 3 as described above, the electronic stethoscope 210 configured to collect a living body sound can sufficiently pick up a living body sound having a low sound pressure level as with the above-described Embodiment 1.

### Embodiment 4

The present Embodiment 4 is a modified embodiment of the above-described Embodiment 3. In the case of the above-described Embodiment 3, the first space S1 and the second space S2 in the internal space S of the housing 216 are directly connected to each other as shown in FIG. 6. On the other hand, in the case of the present Embodiment 4, the first space S1 is indirectly connected to the second space S2. Accordingly, a description will be given of the present Embodiment 4 while focusing on this different feature. Note that constituents that are substantially the same as the constituents of the above-described Embodiment 3 will be denoted by the same reference signs.

FIG. 7 is a cross-sectional view of an electronic stethoscope according to the Embodiment 4.

As shown in FIG. 7, in an electronic stethoscope 310 according to the present Embodiment 4, an internal space S of a housing 316 includes a third space S3 located between a first space S1 and a second space S2. The third space S3 has a cylindrical shape with a uniform transverse sectional shape. The third space S3 is connected to a second space side end S1b of the first space S1 and to a first space side end S2a of the second space S2, respectively, and is defined by an inner wall surface 316e. The inner wall surface 316e is connected to a second space side end of an inner wall surface 316c that defines the first space S1 and to a first space side end of an inner wall surface 316d that defines the second space S2, respectively, and extends therebetween.

Here, the third space S3 shown in FIG. 7 extends linearly. However, the third space S3 is not limited to be of the linear shape.

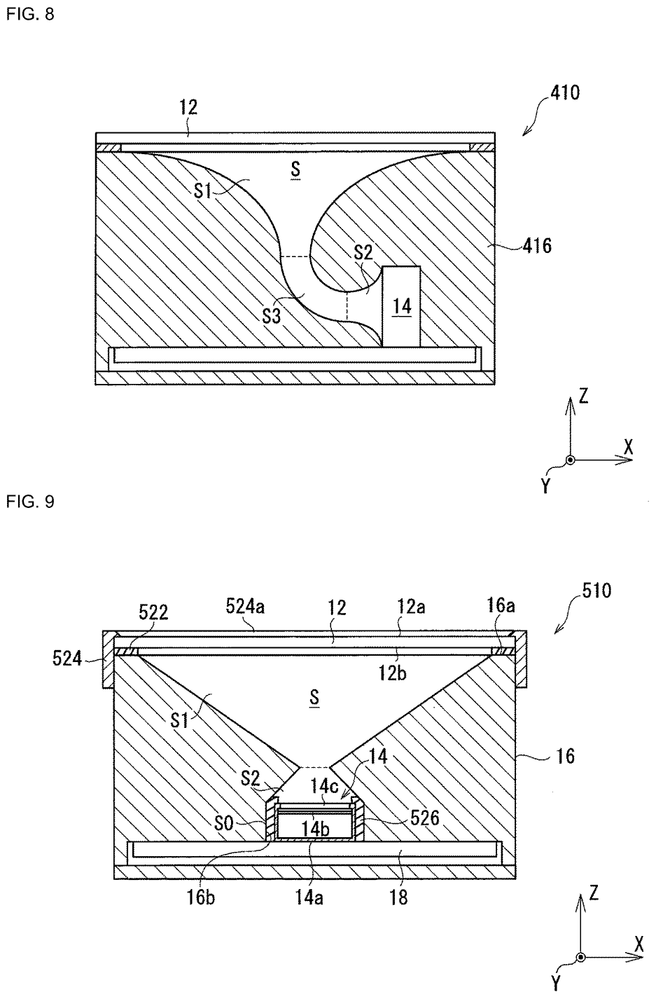

FIG. 8 is a cross-sectional view of an electronic stethoscope according to another example of the Embodiment 4.

As shown in FIG. 8, in an electronic stethoscope 410 according to the other example of the present Embodiment 4, a third space S3 in an internal space S of a housing 416 is a curved space which is bent by 90°.

As described above, according to the third space S3 that may take on various shapes, a positional relation between the diaphragm 12 and the sound sensor 14 can be adjusted without necessarily damaging the effect of amplifying the sound pressure level of the living body sound attributed to the internal space S of the housing. As a consequence, freedom of design of the electronic stethoscope is increased.

According to the present Embodiment 4 as described above, the electronic stethoscopes 310 and 410 configured to collect a living body sound can sufficiently pick up a living body sound having a low sound pressure level as with the above-described Embodiment 1.

### Embodiment 5

The present Embodiment 5 is a modified embodiment of the above-described Embodiment 1, which is capable of further amplifying a sound pressure level of a living body sound. Accordingly, a description will be given of the present Embodiment 5 while focusing on this different feature. Note that constituents that are substantially the same as the constituents of the above-described Embodiment 1 will be denoted by the same reference signs.

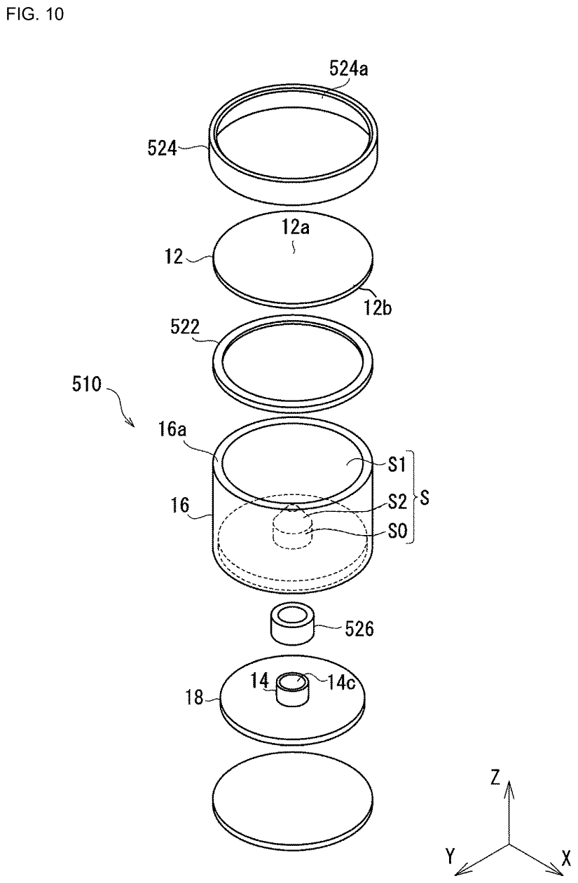

FIG. 9 is a cross-sectional view of an electronic stethoscope according to the Embodiment 5. Meanwhile, FIG. 10 is an exploded perspective view of the electronic stethoscope according to the Embodiment 5.

As shown in FIGS. 9 and 10, an electronic stethoscope 510 according to the present Embodiment 5 is configured to improve a sealing degree of the internal space S of the housing 16 in order to further amplify the sound pressure level of the living body sound.

To this end, in the electronic stethoscope 510 according to the present Embodiment 5, an annular sealing member 522 is disposed between the outer peripheral edge at the second surface 12b of the diaphragm 12 and the end surface 16a of the housing 16 in the first place. The sealing member 522 is formed from an elastic material such as silicone rubber.

Moreover, the sealing member 522 in a compressively deformed state is disposed between the diaphragm 12 and the housing 16. To be more precise, the electronic stethoscope 510 according to the present Embodiment 5 includes an annular cover member 524 for fixing the diaphragm 12 to the end surface 16a of the housing 16. The cover member 524 is attached to the housing 16 in such a way as to cover the outer peripheral edge at the first surface 12a of the diaphragm 12 placed on the end surface 16a of the housing 16 with the sealing member 522 interposed therebetween. When the cover member 524 is attached to the housing 16, the sealing member 522 is compressed in a thickness direction thereof (the z axis direction). In other words, in the state where the cover member 524 is attached to the housing 16, a distance between the second surface 12b of the diaphragm 12 and the end surface 16a of the housing 16 is smaller than the thickness of the sealing member 522.

A space between the diaphragm 12 and the housing 16 is liquid-tightly sealed by disposing the sealing member 522 in the compressively deformed state between the second surface 12b of the diaphragm 12 and the end surface 16a of the housing 16.

In addition, in the electronic stethoscope 510 according to the present Embodiment 5, a tubular sealing member 526 is disposed between the sound sensor 14 and the inner wall surface 16b of sound sensor housing space S0 in the internal space S of the housing 16. The sealing member 526 is formed from an elastic material such as silicone rubber.

Moreover, the sealing member 526 in a compressively deformed state is disposed between the inner wall surface 16b of the housing 16 and the sound sensor 14. To be more precise, when the sealing member 526 is in a natural state, an inside diameter of the cylindrical sealing member 526 is smaller than an outside diameter of the cylindrical sound sensor 14. At the same time, an outside diameter of the sealing member 526 is larger than an inside diameter of the cylindrical sound sensor housing space S0 of the housing 16. Due to the above-mentioned size relations, the sealing member 526 is disposed between the inner wall surface 16b of the housing 16 and the sound sensor 14 in the state of being compressed in radial directions (the x axis direction and the y axis direction).

A space between the sound sensor 14 and the housing 16 is liquid-tightly sealed by disposing the sealing member 526 in the compressively deformed state between the sound sensor 14 and the inner wall surface 16b of the sound sensor housing space S0 in the internal space S of the housing 16.

The internal space S of the housing 16 is substantially set to a hermetically sealed state by using the two sealing members 522 and 526 as described above. Accordingly, the vibration propagated from the diaphragm 12 is propagated toward the sound sensor 14 via the internal space S without necessarily substantially leaking out of the internal space S of the housing 16. As a consequence, the sound pressure level of the living body sound is further increased in the internal space S of the housing 16.

Here, in the electronic stethoscope 510 of the present Embodiment 5, the size of the tubular sealing member 526 is larger than the size of the sound sensor 14 in terms of the direction of extension (the z axis direction) of the sound sensor housing space S0. As a consequence, a portion of the sealing member 526 runs on an end surface of the casing 14a of the sound sensor 14 which is provided with the sound collection port 14c as shown in FIG. 9. As a consequence, the sound sensor 14 is fixed in the direction of extension of the sound sensor housing space S0 by using the sealing member 526.

Meanwhile, the sealing member 522 can be omitted if it is possible to fix the diaphragm 12 to the end surface 16a of the housing 16 without necessarily creating any gaps therebetween, or if it is possible to establish adhesion by using an adhesive, for example. Moreover, the sealing member 526 can be omitted if it is possible to insert the sound sensor 14 into the sound sensor housing space S0 without necessarily creasing any gaps therebetween.

Moreover, the sealing members 522 and 526 according to the present Embodiment 5 can also be used in the electronic stethoscopes 110, 210, 310, and 410 according to the Embodiments 2 to 4.

According to the present Embodiment 5 as described above, the electronic stethoscope 510 configured to collect a living body sound can sufficiently pick up a living body sound having a low sound pressure level as with the above-described Embodiment 1.

Although the present disclosure has been described above by discussing certain embodiments, the embodiments of the present disclosure are not limited thereto.

For example, in the case of the above-described Embodiment 1, the transverse section of the internal space S of the housing 16 has a circular shape. However, the embodiments of the present disclosure are not limited to this configuration. The transverse section of the internal space S of the housing may have a rectangular shape, for example.

Meanwhile, in the case of the above-described Embodiment 1, the first space S1 in the internal space S of the housing 16 has such a shape that the cross-sectional area of the transverse section thereof is gradually decreased from the diaphragm side end Sla toward the second space side end S1b when viewed from any direction as long as it is the direction intersecting with the direction of extension (the z axis direction) of the internal space S (in other words, it is any of the directions including an x axis direction component and a y axis direction component). However, the embodiments of the present disclosure are not limited to this configuration. For example, the first space S1 may have such a shape that the cross-sectional area of the transverse section thereof is gradually decreased when viewed from one of the x axis direction and the y axis direction and that the cross-sectional area of the transverse section thereof is constant when viewed from the other one of these directions.

Moreover, in the case of the above-described Embodiment 1, the second space S2 in the internal space S of the housing 16 has such a shape that the cross-sectional area of the transverse section thereof is gradually increased from the first space side end S2a toward the sound sensor side end S2b when viewed from any direction as long as it is the direction intersecting with the direction of extension (the z axis direction) of the internal space S (in other words, it is any of the directions including the x axis direction component and the y axis direction component). However, the embodiments of the present disclosure are not limited to this configuration. For example, the second space S2 may have such a shape that the cross-sectional area of the transverse section thereof is gradually increased when viewed from one of the x axis direction and the y axis direction and that the cross-sectional area of the transverse section thereof is constant when viewed from the other one of these directions.

Furthermore, in the case of the above-described Embodiment 1, propagation of the vibration from the diaphragm 12 to the sound sensor 14 takes place via the internal space S provided to the housing 16. In other words, the housing 16 is a tubular member that includes an outer peripheral surface constituting an outer surface (a designed surface) of the electronic stethoscope 10 and an inner peripheral surface that defines the internal space S. However, the embodiments of the present disclosure are not limited to this configuration. For example, a tubular member including an internal space to propagate vibration may be provided in a housing that includes an outer surface of an electronic stethoscope.

In conclusion, various aspects of the present disclosure are as follows.

A first aspect is an electronic stethoscope including:
a diaphragm including
   a first surface that comes into contact with a living body, and
   a second surface located on an opposite side of the first surface;
a sound sensor that receives vibration propagated from the diaphragm and converts the vibration into an electric signal; and
a tubular member that includes an inner wall surface to define an internal space in which the vibration is propagated from the diaphragm toward the sound sensor, in which
the inner wall surface of the tubular member includes
   a first inner wall surface that defines a first space on a diaphragm side,
   a second inner wall surface that defines a second space on a sound sensor side, and
   a boundary portion where the first inner wall surface is in contact with the second inner wall surface,
a volume of the first space is larger than a volume of the second space, and
a cross-sectional area of a transverse section of the internal space of the tubular member intersecting with a direction of extension of the internal space is gradually decreased from an end on the diaphragm side toward the boundary portion in the first space and is gradually increased from the boundary portion toward an end on the sound sensor side in the second space.

A second aspect is the electronic stethoscope according to the first aspect, in which the second inner wall surface of the tubular member is convexly curved when viewed in a direction intersecting with the direction of extension of the internal space and defines the second space.

A third aspect is the electronic stethoscope according to the second aspect, in which a curvature radius of the second inner wall surface is smaller at a portion close to the end on the sound sensor side in comparison between a portion close to an end on a first space side and the portion close to the end on the sound sensor side.

A fourth aspect is the electronic stethoscope according to any one of the first to third aspects, in which the first inner wall surface of the tubular member is convexly curved when viewed in a direction intersecting with the direction of extension of the internal space, and defines the first space.

A fifth aspect is the electronic stethoscope according to the fourth aspect, further including:
an annular spacer member provided between an outer peripheral edge at the second surface of the diaphragm and an end surface of the tubular member and configured to locate the second surface of the diaphragm away from the first inner wall surface of the tubular member.

A sixth aspect is the electronic stethoscope according to any one of the first to fifth aspects, in which the inner wall surface of the tubular member includes a third inner wall surface that is connected to an end on a second space side of the first inner wall surface defining the first space and to an end on a first space side of the second inner wall surface defining the second space, respectively, and defines a third space having a uniform transverse sectional shape.

A seventh aspect is the electronic stethoscope according to any one of the first to sixth aspects, in which
the internal space of the tubular member includes a sound sensor housing space connected to an end on the sound sensor side of the second space and configured to house the sound sensor, and
the electronic stethoscope further includes a tubular sealing member disposed in a compressively deformed state between an inner wall surface of the sound sensor housing space and the sound sensor.

An eighth aspect is the electronic stethoscope according to the seventh aspect, in which a size of the tubular sealing member is larger than a size of the sound sensor in terms of a direction of extension of the sound sensor housing space of the tubular member.

A ninth aspect is the electronic stethoscope according to any one of the first to eighth aspects, further including:
an annular sealing member disposed in a compressively deformed state between an outer peripheral edge at the second surface of the diaphragm and an end surface of the tubular member.

What is claimed is:

1. An electronic stethoscope comprising:
a diaphragm having a first surface configured to contact a living body, and a second surface located on an opposite side of the first surface;
a sound sensor configured to receive vibration propagated from the diaphragm and to convert the vibration into an electric signal; and
a tubular member having an inner wall surface that defines an internal space in which the vibration is propagated from the diaphragm toward the sound sensor,
wherein the inner wall surface of the tubular member includes:
a first inner wall surface that defines a first space on a diaphragm side,
a second inner wall surface that defines a second space on a sound sensor side, and
a boundary portion where the first inner wall surface is in contact with the second inner wall surface,
wherein a volume of the first space is larger than a volume of the second space, and
wherein a cross-sectional area of a transverse section of the internal space of the tubular member intersecting with a direction of extension of the internal space gradually decreases from an end on the diaphragm side toward the boundary portion in the first space and gradually increases from the boundary portion toward an end on the sound sensor side in the second space.

2. The electronic stethoscope according to claim 1, wherein the second inner wall surface of the tubular member is convexly curved when viewed in a direction intersecting with the direction of extension of the internal space, and defines the second space.

3. The electronic stethoscope according to claim 2, wherein a curvature radius of the second inner wall surface is smaller at a portion close to the end on the sound sensor side than a portion close to an end on a first space side and the portion close to the end on the sound sensor side.

4. The electronic stethoscope according to claim 1, wherein the first inner wall surface of the tubular member is convexly curved when viewed in a direction intersecting with the direction of extension of the internal space, and defines the first space.

5. The electronic stethoscope according to claim 4, further comprising:
an annular spacer between an outer peripheral edge at the second surface of the diaphragm and an end surface of the tubular member, and configured to locate the second surface of the diaphragm away from the first inner wall surface of the tubular member.

6. The electronic stethoscope according to claim 1, wherein the inner wall surface of the tubular member includes a third inner wall surface that is connected to an end on a second space side of the first inner wall surface defining the first space and to an end on a first space side of the second inner wall surface defining the second space, and defines a third space having a uniform transverse sectional shape.

7. The electronic stethoscope according to claim 1,
wherein the internal space of the tubular member includes a sound sensor housing space connected to an end on the sound sensor side of the second space and configured to house the sound sensor, and
the electronic stethoscope further comprises a tubular sealing member disposed in a compressively deformed state between an inner wall surface of the sound sensor housing space and the sound sensor.

8. The electronic stethoscope according to claim 7, wherein a size of the tubular sealing member is larger than a size of the sound sensor in a direction of extension of the sound sensor housing space of the tubular member.

9. The electronic stethoscope according to claim 1, further comprising:
an annular sealing member disposed in a compressively deformed state between an outer peripheral edge at the second surface of the diaphragm and an end surface of the tubular member.
